(19) United States
(12) Patent Application Publication
(10) Pub. No.: US 2013/0317402 A1
(43) Pub. Date: Nov. 28, 2013

(54) Expandable device for immobilizing fractures, severe sprains, or soft tissue injuries

(71) Applicant: Biofab, Inc., West Palm Beach, FL (US)

(72) Inventors: Henry Pong, Boynton Beach, FL (US); Edward W. Sandall, West Palm Beach, FL (US)

(21) Appl. No.: 13/901,177

(22) Filed: May 23, 2013

Related U.S. Application Data
(60) Provisional application No. 61/650,515, filed on May 23, 2012.

(51) Int. Cl. A61F 5/01

(52) U.S. Cl. CPC A61F 5/0111
USPC 602/6

(57) ABSTRACT

The present invention provides an easily transportable immobilization device and system designed to immobilize fractures, severe sprains or soft tissue injuries within a body structure. The device generally contains a shaped body having one or more portions constructed and arranged to fit onto a leg or arm having a fracture, severe strain or soft tissue injury. The device utilizes a multilayer construction which allows for the delivery of the device to an individual in need in a compressed state. Activation allows a portion of the device to return to the non-compressed state, uniformly expanding outwardly, both longitudinally and radially. As the device expands, it surrounds and contacts the injured body structure, thereby applying a pressure as well as preventing lateral movement. The injured body structure is completely immobilized and secured until proper evaluation and care can be performed.

### CROSS REFERENCE TO RELATED APPLICATIONS

In accordance with 37 C.F.R 1.76, a claim of priority is included in an Application Data Sheet filed concurrently herewith. Accordingly, under 35 U.S.C. §119(e), 120, 121, and/or 365(c) the present invention claims priority to Application No. 61/650,515, entitled “EXPANDABLE DEVICE FOR IMMOBILIZING FRACTURES, SEVERE SPRAINS, OR SOFT TISSUE INJURIES”, filed on May 23, 2012. The content of the above referenced application is herein incorporated by reference in its entirety.

### FIELD OF THE INVENTION

The present invention relates to medical devices and systems that maintain one or more portions of a human extremity in a supported and fixed position, and more particularly to an easily transportable, expandable immobilization device and system designed to immobilize fractures, severe sprains or soft tissue injuries.

### BACKGROUND OF THE INVENTION

Fractures, severe sprains, and soft tissue injuries involving the upper and lower limbs of humans are common. Most of these injuries are a result of some sort of trauma event, including sports related injuries, car accidents, or work related accidents. Natural disasters, including earthquakes or tsunamis, often result in a large number of casualties, many of which individuals suffer from one or more fractures to the arms or lower leg. One of the problems associated with any injury is the need to obtain proper medical treatment in a timely manner. While fractures, severe sprains, and soft tissue injuries are generally not life threatening, the inability to receive proper treatment often results in severe secondary complications.

Once the patient obtains proper medical analysis, the patient can undergo corrective measures, i.e. surgical intervention or non-surgical, stabilization, elevation, rest and ice treatment. However, for many individuals, particularly in poor countries without complex transportation systems or areas hit by a disaster, i.e. tornadoes, tsunamis, where large numbers of injuries are reported and damage to infrastructure makes getting to those individuals difficult, improper immobilization of an extremity injury, such as fractures, can be disastrous. Should a fracture not receive proper immobilization, the patient risks the effects of the bone flopping around, swelling, pain, and/or the immobilized bone puncturing the skin, all of which can lead to serious complications. In the case of skin puncturing, such actions can lead to serious infection and often times result in amputation. If an easy to manufacture, simple to transport and apply device could be delivered to the site and immobilize the fracture, such complications may be reduced. In cases where professionals may not be able to get to the individuals in time, the device would provide a means for immobilization. Even if the immobilization is not proper, and the fracture heals improperly, corrective surgery may be employed to fix the problem at a later time. However, complications such as amputation would be avoided.

### DESCRIPTION OF THE PRIOR ART

Devices for maintaining a human extremity, or portions thereof, in a fixed position are known in the art. Such devices include casts, splints, or elevation devices that are generally designed to fix a human structure, such as the foot, ankle, or toe in place. For example, U.S. Pat. No. 5,385,534 describes a splint and kit for assembling the splint. The splint is described as including a plurality of components which, when assembled, provide a device which conforms to the upper portion of the leg. The components include a support member, a pad for cushioning the support member against the leg, and one or more straps for securing the support member to the body. A pair of flexible members for fixedly retaining the flexion angle of the support member is also described.

U.S. Pat. No. 5,449,339 describes a heel supporting protective boot. The boot is described as being useful for bed patients. The boot comprises a formed flexible, compressible foam material and leg supporting portions for supporting a patient's foot. While the boot is described as being compressible, the device does not expand to apply pressure in the manner as described herein. Moreover, repeated compression can result in shape deformity. The size of the device makes it difficult to ship and store.

U.S. Pat. No. 5,078,128 discloses a leg walker for stabilizing a lower leg of a patient. The injured limb is placed in a removable inner ankle bootie whose interior is partially covered with inflatable support bladders.

U.S. Pat. No. 5,603,692 describes a foot drop leg splint which is described as useful for stabilizing a patient's foot. The splint is described as having a rigid support frame with a contoured foam insert. Removable straps are used to hold the splint onto the patient. The leg splint is formed of a unitary piece and therefore makes it difficult to ship. Moreover, given the size of the device, storage of the device can be problematic.

U.S. Pat. No. 5,577,998 discloses a walking brace having inflatable envelopes to help maintain firm contact between the limb and the brace.

U.S. Pat. No. 7,524,296 discloses a vacuum orthopedic brace and method that includes the use of an inner sleeve or liner, and at least one rigid member. The rigid member is described as including a fabric liner or other porous material to act as a wick, and a gasket surrounding the wick material. A valve is preferably provided in the rigid member for drawing air from between the rigid member and the liner, i.e. to create a vacuum. Furthermore, an external, internal or integrated chamber or rigid reservoir is preferably included, and is in communication with the wick filled area between the rigid member and liner, to hold a vacuum.

U.S. Publication No. 2005/0145256 discloses an orthopedic walker which has a rigid external shell, a pliant internal boot positioned in the external shell, and a deformable insert positioned within the internal boot. The internal boot is described as being sized to enclose a lower extremity of a user. The deformable insert has a heel segment including a fully-encircled heel opening. The deformable insert freely rests within the internal boot and is sized and positioned to receive the heel of the lower extremity within the heel opening. However, the deformable insert is sufficiently undersized to maintain a continuous exposed surface extending between a top surface of the foot and a front surface of the ankle.

U.S. Pat. Nos. 6,551,280; 7,396,345 and 7,691,084 all show envelopes with vacuum foam chambers that can be used to apply pressure to foot or leg when vacuum is released. The disclosure is described as being directed to therapeutic devices that provide support or pressure to tissue. The device is further described as containing a foam material that is capable of being contracted from its relaxed state to an evacuated state. The material applies pressure to the tissue of a patient when the material is in its relaxed state (expanded). Reduction of pressure by application of a vacuum results in a contracted state of the device, so that the device can be applied or delivered to a patient. Release of vacuum results in a return to the relaxed state, thus providing a maximum desirable pressure to tissue and avoiding the possibility of over-pressure which can result in tissue damage or necrosis. The device requires active input, requiring the use of a vacuum/air pump for operation. Such devices are not as effective in emergency situations or treating individuals in remote areas or those areas hit by a large man-made or natural disaster.

Therefore, what is needed in the art is an improved device that is easy to make, lightweight for transport, functions with a minimal number of components, and can be used by first responders, or individuals in situations without first responders, to immobilize injuries and prevent complications.

### SUMMARY OF THE INVENTION

The present invention provides an easily transportable immobilization device and system designed to immobilize fractures, severe sprains or soft tissue injuries within a body structure of an animal. The device generally contains a expandable, shaped body having one or more portions constructed and arranged to contour the shape of for example an extremity or limb such as a leg or arm having a fracture, severe sprains or soft tissue injuries. The device may utilize a multilayer construction, with at least one layer containing a compressible and expandable material in a compressed state. Activation allows a portion of the device to return to the non-compressed state, uniformly expanding outwardly, both longitudinally and radially. As the device expands, it surrounds and contacts the injured body structure, thereby applying a pressure as well as preventing lateral movement. The injured body structure is completely immobilized and secured until additional evaluation and care can be performed

Accordingly, it is an objective of the present invention to provide a device for immobilizing fractures, severe sprains or soft tissue injuries.

It is a further objective of the present invention to provide an immobilizing device that can be easily and quickly secured to an injured body part.

It is yet another objective of the present invention to provide an immobilizing device that can be easily stored using minimal space.

It is a still further objective of the present invention to provide an immobilizing device that is lightweight.

It is yet another objective of the present invention to provide an immobilizing device that functions using a minimal number of components.

It is a further objective of the present invention to provide an immobilizing device which provides temporary protection from harm created by an immobilized injury as well as decreases swelling, bleeding and pain until proper care is obtained.

It is yet another objective of the present invention to provide first responders the ability to easily and quickly apply an immobilizing device to an injured person.

It is a still further objective of the present invention to provide a device that minimizes the risk associated with improperly immobilizing fractures, severe sprains or soft tissue injuries.

It is a further objective of the present invention to provide an immobilizing device having a multilayer construction.

It is yet another objective of the present invention to provide an immobilizing device having a multilayer construction multilayer construction, with at least one layer containing a compressible and expandable material in a compressed state.

It is a still further objective of the present invention to provide an immobilizing device having a multilayer construction which can be activated to surround and contact an injured body structure, thereby applying a pressure as well as preventing lateral movement.

It is yet another objective of the present invention to provide an immobilizing device having a multilayer construction which can be activated to allow a portion of the device to return to a non-compressed state.

It is a further objective of the present invention to provide an immobilizing device having a multilayer construction which can be activated to allow a portion of the device to return to a non-compressed state, uniformly expanding outwardly, both longitudinally and radially.

Other objectives and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is a perspective view of an illustrative example of an expandable device for immobilizing fractures within an extremity in accordance with the present invention;

FIG. 2 a perspective view of an illustrative example of an expandable device for immobilizing fractures within an extremity shown in FIG. 1 with securing features;

FIG. 3 is a left side view of the expandable device for immobilizing fractures within an extremity shown in FIG. 1;

FIG. 4 is a right side view of the expandable device for immobilizing fractures within an extremity shown in FIG. 1;

FIG. 5A is a partial view of an illustrative example of the multilayer construction of the expandable device for immobilizing fractures within an extremity in accordance with the present invention;

FIG. 5B is a longitudinal cross sectional view taken along lines 5B-5B of FIG. 5A, illustrating the components of the multilayer construction of the expandable device for immobilizing fractures within an extremity shown in FIG. 5A in the compressed state;

FIG. 5C illustrates the components of the multilayer construction of the expandable device for immobilizing fractures within an extremity shown in FIG. 5A in the extended, non-compressed state;

FIG. 5D illustrates the components of the multilayer construction of the expandable device for immobilizing fractures within an extremity shown in FIG. 5A in the extended, non-compressed state with an absorbent material;

FIG. 6A is a partial view of an alternative embodiment of the multilayer construction of the expandable device for immobilizing fractures within an extremity in accordance with the present invention;

FIG. 6B is a longitudinal cross sectional view taken along lines 6B-6B of FIG. 6A, illustrating the components of the multilayer construction of the expandable device for immobilizing fractures within an extremity shown in FIG. 6A in the compressed state;

FIG. 6C illustrates the components of the multilayer construction of the expandable device for immobilizing fractures within an extremity shown in FIG. 6A in the extended, non-compressed state;

FIG. 6D illustrates the components of the multilayer construction of the expandable device for immobilizing fractures within an extremity shown in FIG. 6A in the extended, non-compressed state with an absorbent material;

FIG. 7 is a cross sectional view taken along lines 7-7 of FIG. 4;

FIG. 8 is a perspective view of an alternative embodiment of an expandable device for immobilizing fractures within an extremity in accordance with the present invention;

FIG. 9 is an exploded view of the alternative embodiment of an expandable device for immobilizing fractures within an extremity illustrated in FIG. 8;

FIG. 10 is a perspective view of a sole support member;

FIG. 11 is a side view of the sole support device;

FIG. 12 is a partial view of the expandable device for immobilizing fractures within an extremity illustrated in FIG. 8 with securing members;

FIG. 13 is a top view of an illustrative example of the expandable device for immobilizing fractures within an extremity illustrated in FIG. 1 or 8, having a planar reinforcement structure;

FIG. 14 is a top view of an illustrative example of the expandable device for immobilizing fractures within an extremity illustrated in FIG. 1 or 8, having a geometrically configured, generally triangular shaped reinforcement structure;

FIG. 15 is a top view of the expandable device for immobilizing fractures within an extremity, with a portion of the device shown in the compressed state, with leg inserted therein;

FIG. 16 is a top view of the expandable device for immobilizing fractures within an extremity, with a portion of the device shown in the expanded, non-compressed state, with leg inserted therein;

FIG. 17 is a side view of the expandable device for immobilizing fractures within an extremity in accordance with the present invention in the compressed state and inserted within a package;

FIG. 18 is a front view of the expandable device for immobilizing fractures within an extremity in accordance with the present invention in the compressed state and removed from the package, prior to being positioned over a structure requiring immobilization.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

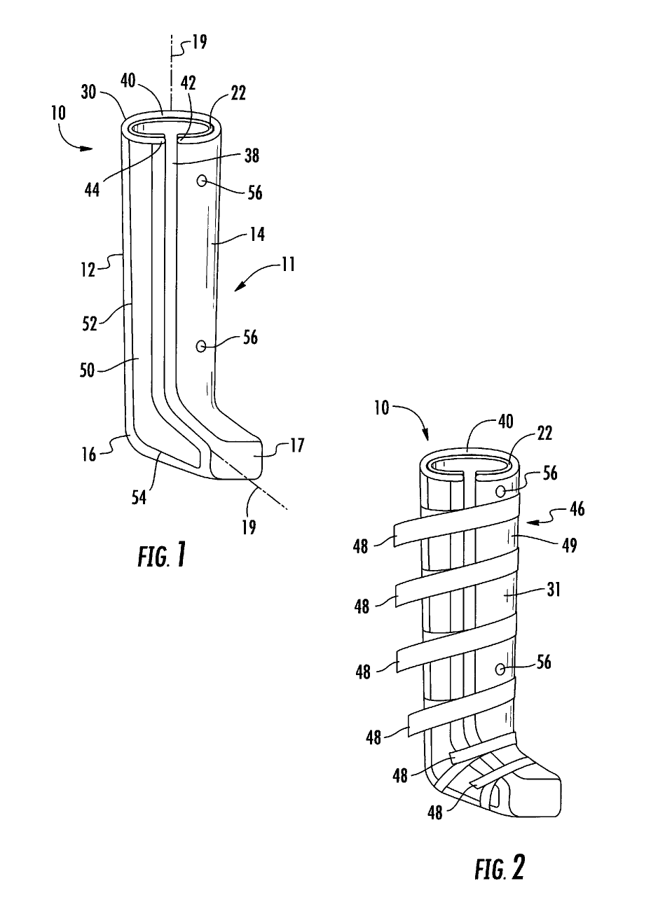

Referring to FIGS. 1-4, an expandable device for immobilizing fractures within an animal extremity, limb or jointed appendage of a animal body, referred generally as 10, is shown. While the present invention is described in use for a human, the device 10 can be used in other animals, such as but not limited to dogs, horses, monkeys. The expandable, immobilizing device 10 is designed to be applied easily and quickly by trained and untrained personnel. As such, the expandable, immobilizing device 10 may be supplied and generally preformed into a desired shape prior to use or may be supplied as a sheet which can be folded to the desired shape. The expandable, immobilizing device 10 requires no additional features or requirements, such as an external air/vacuum source or materials to create hard casts or splints. As a result, the expandable, immobilizing device 10 can be used in any environment, including remote areas, aquatic environments, or large urban areas.

The expandable and immobilizing device 10 is adapted to immobilize fractures, severe sprains, and soft tissue injuries. In an illustrative example, the expandable, immobilizing device 10 comprises a body 11 having first elongated portion illustrated herein as a first long arm 12 having a generally elongated tubular portion 14 positioned in a first direction and a second elongated portion illustrated herein as a second short arm 16 aligned in a second direction. The short arm position may be sized and shaped to configure to a foot and may have, preferably, an open end 17 or a closed end. Alternatively, the first long arm 12 and the second short arm 16 may be sized at or about the same lengths. The body 11 has a non-linear longitudinal center line 19 that contours the shape of an extremity of an animal, such as a human arm, or a human, horse or dog leg. As such, the non-linear longitudinal center line 19 can extend along different planes and include one or multiple curvatures. Preferably, the first long arm 12 and the second short arm 16 form a contoured configuration having curvature or an angled connection, which forms a generally L-shaped configuration. While not illustrated, the main body 11 may include additional curvature or arms to allow for contouring the shape of the main body to fit from the shoulder to the finger tips with contour regions at the elbow and/or wrist if needed. The shape of the first long arm 12 and/or the second short arm 16 may also include curvature and/or rounded portions. Preferably, the angle forms about a 90 degree connection, however, obtuse or acute angles may be utilized. The L-shaped configuration allows the expandable, immobilizing device 10 to be adapted to be insertable over a lower extremity, i.e. the leg and foot, and an upper extremity, i.e. an arm. For example, should an injured individual require immobilization of his/her leg, the device can be inserted over the leg so that the generally elongated tubular portion 14 is positioned at or near the knee and the short arm portion 16 rests on or along the foot and toes. Should the expandable and immobilizing device 10 require use for immobilization of an arm, the short arm portion of the L-shaped expandable, immobilizing device 10 may be designed to fit the area extending from the elbow to the shoulder when the arm is in bent configuration, with the generally elongated tubular portion 14 surrounding the forearm and/or the fingers.

While the present invention is described in an illustrative, albeit preferred embodiment, the expandable and immobilizing device 10 need not be shaped in an L-configuration. Other shapes, such as a substantially C-shape, or substantially tubular shape, which immobilizes a body portion may be used. Additionally, the length of the expandable, immobilizing device may be varied depending on the need and placement of the individual. Such size variation could include larger devices for tall adults and smaller devices for shorter adults or children.

As described previously, the expandable, immobilizing device 10 is designed so that a portion of the device expands from a first compressed position to a second extended, non-compressed position without the need for external vacuum and/or air devices. The device further provides a stable and secure immobilization system without requiring the need for additional components, as required with splints or casts. To achieve such functionality, the expandable, immobilizing device 10 preferably comprises a multi-layer construction. The expandable, immobilizing device 10 may be constructed of a double walled configuration, having a first outer wall 18 and a second inner wall 20, see FIGS. 5A-5C. The outer wall 18 forms the exterior surface while the inner wall 20 is designed to engage and/or conform to the shape of the body structure, i.e. the leg or arm, inserted therein. Placed between the first outer wall and the second inner wall 20 is a compressible and expandable, or self inflating, material 22 in the compressed state form 24, see FIG. 5B. The first outer wall 18 and the second inner wall 20 are made of materials that allow for the compressible materials to be compressed, such as under a vacuum, and which provides enough strength to support and immobilize the inserted arm or leg. The material may be, for example, pliable non-porous materials such as polyester or polyethelene rubber.

The compressible and expandable material 22 may be any suitable natural or synthetic compressible material, which may include open-celled cellular materials, such as natural and synthetic sponges, sponge rubber, polyurethane, polyester, polyethelene, cellulose foam, or may include other compressible materials such as super compressible foam-like carbon nanotube films, see Science, 25 Nov. 2005, Vol. 310. Preferably, the compressible and expandable material 22 is a foam filler that is sealed within the space between the first outer wall 18 and the second inner wall 20 through a vacuum to a desired immobilizing device compressed thickness defined by the distance 26 between outer wall 18 and the second inner wall 20. The compressible and expandable material 22 must also exhibit the property of returning to its non-compress state, 28 see FIG. 5C, when the vacuum conditions are removed.

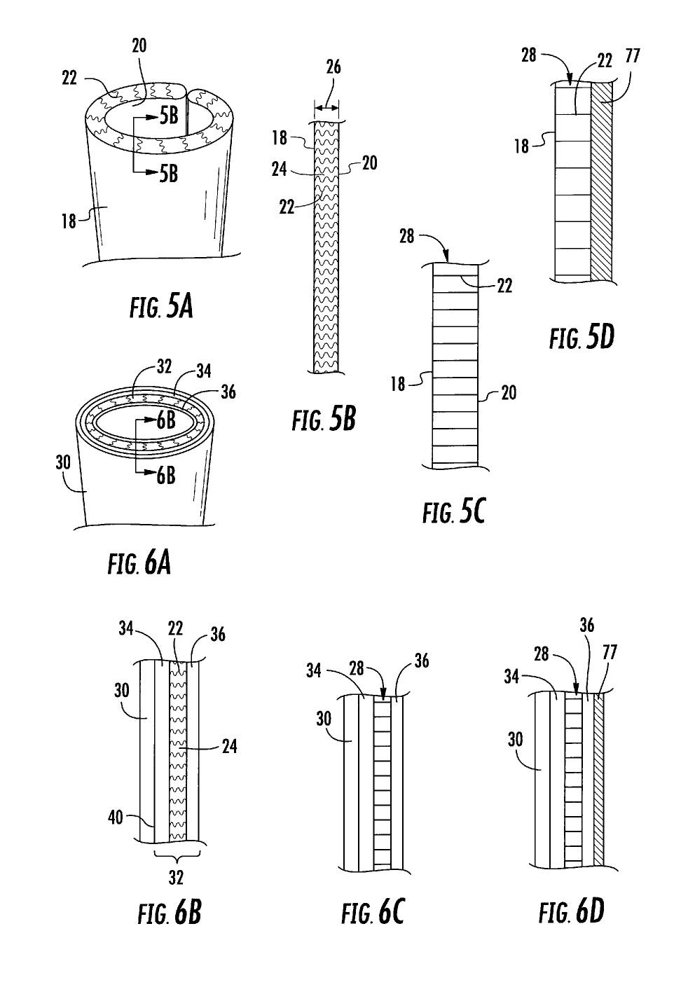

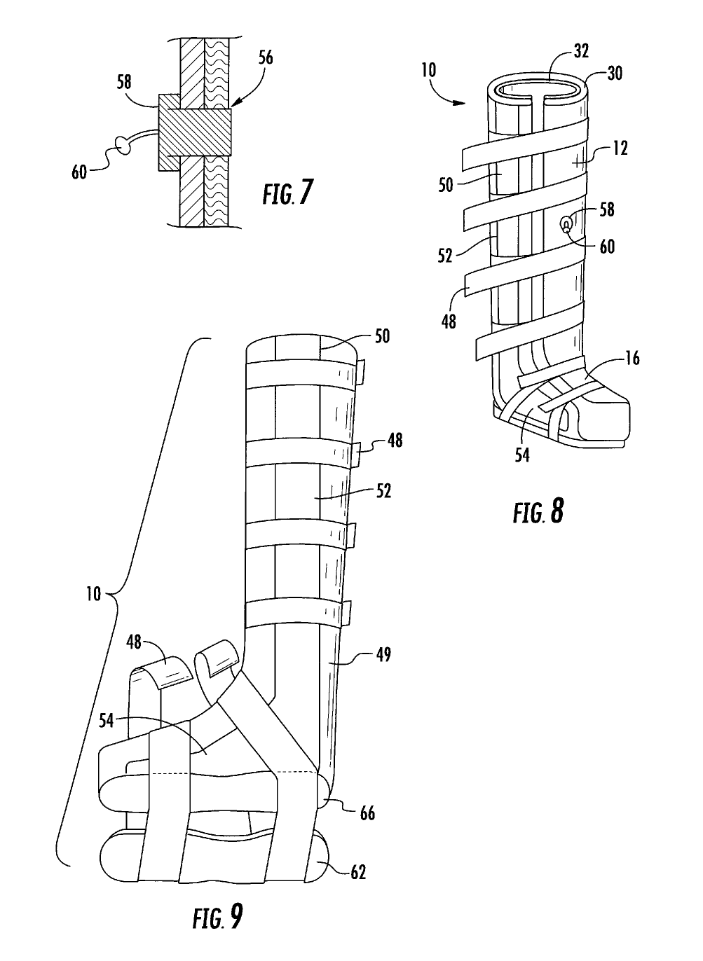

In an alternative embodiment, the immobilizing device 10 contains a single wall 30 and a compressible material pouch secured thereto, see for example FIGS. 6A-6C. The compressible material pouch 32 is made of two sheets 34 and 36 separated by the compressible and expandable material 22 in the compressed state form 24. The two sheets 34 and 36 can be made of pliable, non-porous materials such as polyester or polyethelene rubber. The sheets 34 and 36 are sealed together using any bonding technique known to one of skill in the art, including but not limited to chemical bonding such as the use of adhesives or sealing techniques such as thermal bonding.

The compressible material pouch 32 may then be chemically sealed to the inner surface 40 of the wall 30 using chemical means such as adhesives. Because the compressible material pouch 32 is made from pliable material, the compressible pouch 32 is designed to withstand the expansion of the compressible and expandable material 22 in the non-compressed state 28, see FIG. 6A. Accordingly, there must be enough of the sheet material to withstand expansion or the sheet must be made of materials that can expand to the desired position without damage. In addition to the pliable nature, the sheets 34 and 36 are designed with enough material to ensure that the compressible and expandable material 22 can expand to its non-compressed size and shape. Alternatively, the sheet 36 may be made to break apart, allowing the compressible and expandable material 22 to expand to full capacity and be in direct contact with the human structure inserted therein.

Referring back to FIG. 1 or 2, the expandable, immobilizing device 10 is illustrated as having a single wall 30 construction having a predetermined thickness with the compressible and expandable material 22 secured to the inner wall surface 40. The single wall 30 contains a slotted opening or an aperture 38 which is sized and shaped to allow the device 10 to be easily fitted to an injured human structure, such as an arm or leg. Accordingly, the slotted opening or an aperture 38 is sized to allow for insertion of an animal extremity therein and having a length that contours at least a portion of the expandable body. The slotted opening 38 is illustrated as having an opening size defined by the distance between a first end 42 and a second end 44 and having a length which runs along the entire length of the non-linear longitudinal center line 19, i.e. along the first long arm 12 and the second short arm 16 to allow maximum ease in inserting the expandable, immobilizing device 10 over the human structure. The length and distance of the slotted opening or an aperture 38 can be varied depending on the need of the user. For instance, the second short arm 16 may not contain any slotted opening or an aperture 38 or the slotted opening or an aperture 38 may extend partially within the length of the short arm 16.

Securing members 46, illustrated herein as straps 48, are attached to one or more portions of the outer surface 49 of the expandable, immobilizing device 10. The straps 48 can be secured to the expandable, immobilizing device 10 using adhesion methods, glue to tape, or other mechanisms such as loop and fastener means, for example VELCRO. Alternatively, the straps may not be secured directly to the device. The straps 48 are preferably made of nylon material, but may be made of any other materials, and designed so that one portion of the strap 48 can removably secure to another portion. Such functionality can be accomplished by the use of loop and fastener means, such as VELCRO. Other securing members 46, individually or in combination, may be used and positioned at various places on the expandable, immobilizing device 10, including at or near the first end 42 and the second end 44, extending along the entire length of the expandable, immobilizing device 10. The securing members 46 may include, but are not limited to buttons, snaps, zippers, or loop and fastener securing means, such as VELCRO. Additionally, the straps 48 may contain snaps or buttons to fasten one portion of the strap to another.

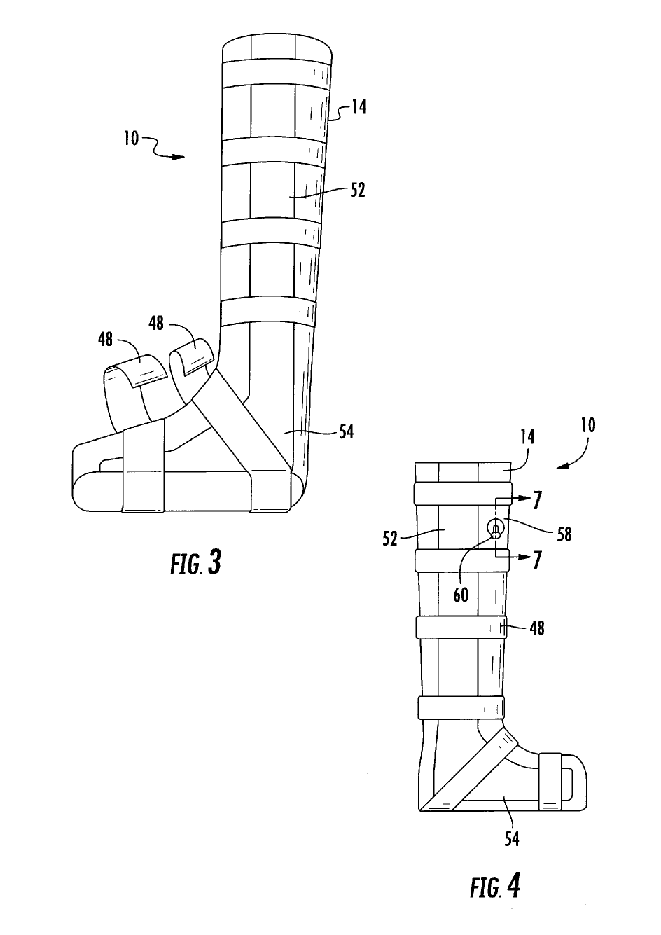

One or more reinforcement structures 50 are attached to the outer surface 49 of the expandable, immobilizing device for providing support and stability. The reinforcement structure 50 is preferably designed to mirror the shape of the expandable, immobilizing device 10 and has a length that mirrors, partially or completely, the length of the non-linear longitudinal center line 19. The reinforcement structure 50 therefore can be sized and shaped to contour at least a portion of said expandable body. As such, the reinforcement structure 50 may include a first stabilizing structure portion 52 having a length that is substantially the same size as the generally elongated tubular portion 14. A second stabilizing structure portion 54 is designed to mirror the shape of the short arm portion 16. As shown in FIGS. 3 and 4, the stabilizing structure contains a substantially L-shaped configuration.

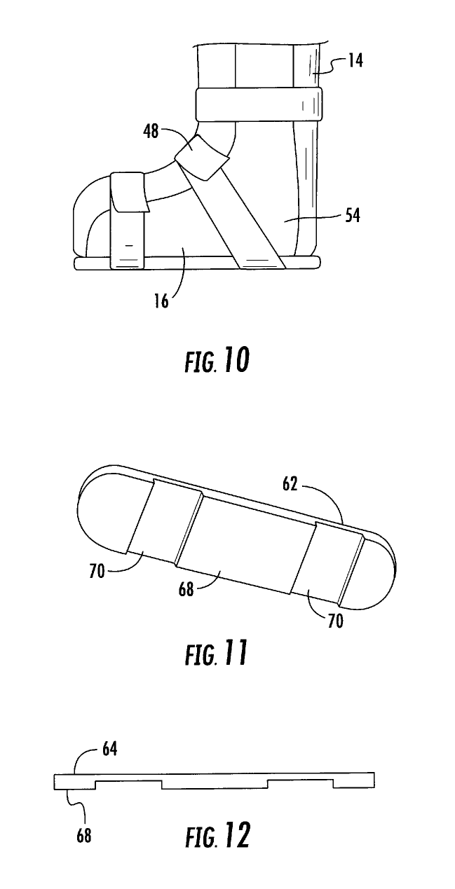

The expandable, immobilizing device 10 may contain one or more covered compression or release openings 56 which may be used to provide a means for allowing the compressible and expandable material 22 to expand from a compressed state to an expanded, non-compressed state and a mechanism to from the compressible and expandable material 22 in a compressed state. For example, the covered compression or release opening 56 may be sealed with a plug 58, see FIGS. 4 and 7. The plug 58 is sized and shaped to seal the opening 56. The plug 58 contains a pull device, such as tab 60, which when activated, i.e. pulled, removes the plug, thereby removing the vacuum seal. The opening 56 may be covered by a material which can be punctured by a sharp object, thereby breaking the vacuum seal of the compressible material pouch 32. Alternatively, the first wall and/or the compressible material pouch 32 may be made of a puncturable material so that the user may simply insert a sharp object along any portion of the wall 30 or expandable, immobilizing device 10. Other means of breaking the vacuum may be utilized, and include the use of perforations (not illustrated) positioned along the top circumference of the long arm 12 so that the user can tear apart a portion of the device at the perforation sites.

FIGS. 8-10 illustrate an alternative embodiment of the expandable, immobilizing device 10. The expandable, immobilizing device 10 contains all the same features as described above. The expandable, immobilizing device 10 in accordance with the embodiment illustrated in these figures is specifically used for immobilizing the foot, and includes a mechanism for enhanced stabilization and support of the foot. Referring to FIGS. 8 and 9, the expandable, immobilizing device 10 contains a bottom support structure, illustrated herein as a rubber or neoprene sole 62. The sole 62 contains a first surface 64 which secures to the bottom surface 66 of the device 10 and a second surface 68 which contacts the ground, see FIGS. 9, 11 and 12. The sole 62 can be permanently fixed to the bottom surface 66 of the expandable, immobilizing device 10 through techniques known to one of skill in the arts, such as through adhesives. Alternatively, the sole 62 can be attached and secured to the bottom of the expandable, immobilizing device 10 through the use of nylon straps 48 and fastener members, such as VELCRO (not shown). If secured through the use of straps 48, the sole 62 may contain one or more grooves or recessed portions 70 sized and shaped to receive and retain the nylon straps 48.

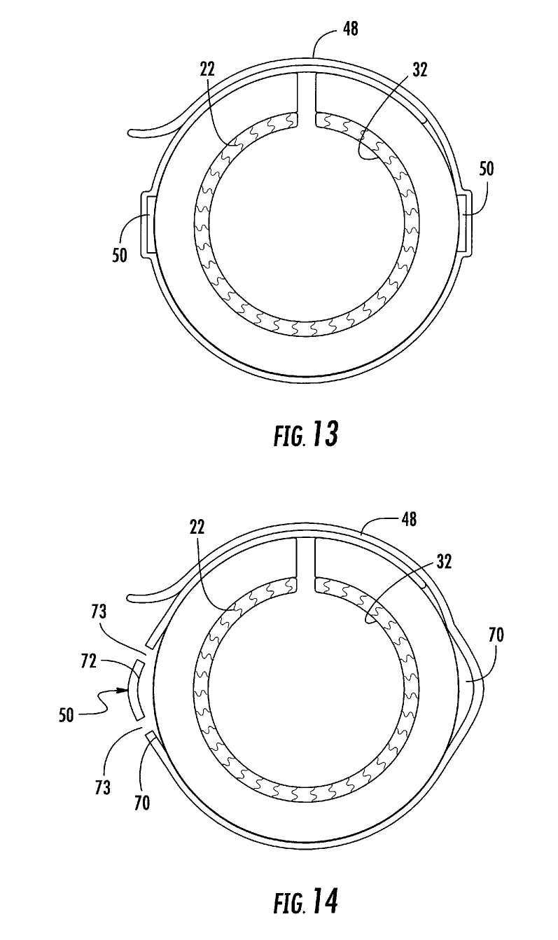

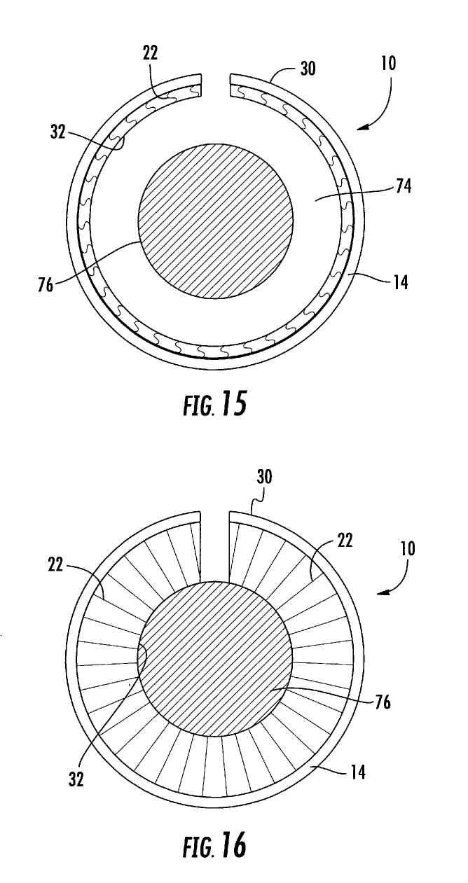

In a similar manner as described previously, the device 10 contains one or more L-shaped reinforcement structures 50, which is preferably made from a rigid plastic material. The reinforcement structure 50 attaches to the outer surface 49 by adhesion mechanisms, such as using chemical adhesives or through the use of double sided tape. As shown in FIGS. 9 and 13, the L-shaped reinforcement structures 50 are generally flat, planer structures. To provide additional support, particularly more side to side as well as back to front stability, the reinforcement structures 50 can contain a three dimensional configuration. As illustrated in FIG. 14, the reinforcement structures 50 contain a generally triangular shape 70. The triangular shape may form equal sides, but need not, and have a pointed or non-pointed apex region 72. Several slotted regions 73 within the triangular shape 70 may be used for insertion of VELCRO straps therein to secure the device to the inserted structure.

Additional features of the present invention are further illustrated by a description of its use in immobilizing a leg. The design of the expandable, immobilizing device 10 provides a readymade, lightweight and inexpensive device for immobilizing fractures, severe sprains, and soft tissue injuries of the extremities. Because of the design features, the expandable, immobilizing device 10 finds particular use in natural or man-made disasters, allowing rescue teams with the ability to apply immobilizing devices that do not require a large number of components to be functional. Because it is easily transportable, quick to install, i.e. does not require surgical wrapping, and does not require other equipment for full functionality, i.e. pumps, it can be used in all types of regions and all types of disasters. While the expandable, immobilizing device 10 may find use in rescue situations, it can be used in hospital settings or at physician offices.

As an illustrative example, the expandable, immobilizing device 10 is used to secure a fracture below the knee of an injured person. Upon encountering the injured individual, the first responder or physician secures the L-shaped device to the individual by placing the second arm 16 over the foot. The longer first arm 14 is positioned over the lower leg. The first responder secures the expandable, immobilizing device 10 to the individual by securing the nylon straps 48 to the desired position. Once secured, a space 74 between the compression material 22 and the inserted leg 76 exists, see FIG. 15.

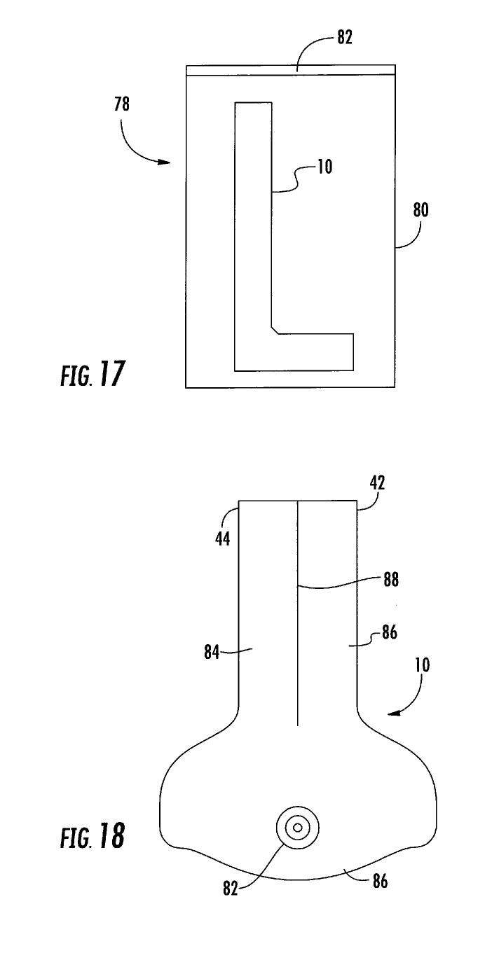

To activate the device, the first responder inserts a sharp object through either the opening 56 or anywhere through the puncturable material that makes up the expandable, immobilizing device 10, or by pulling on the tab 60, thereby releasing plug 58. In any manner, the vacuum is released and the compressible and expandable material 22 within the compressible material pouch 32, which is originally in the compressed state, returns to the non-compressed state. As the compressible and expandable 22 expands, it uniformly expands outwardly in a longitudinal and radial direction. As the compressible and expandable material 22 expands, it surrounds and contacts the leg 76, see FIG. 16, thereby applying a pressure to the leg as well as preventing lateral movement. The leg 76 is now immobilized and secured until proper evaluation and care can be performed.

Both the outside surface 49 of the device, as well as one or both of the sheets 34 and 36 that makes up the compressible material pouch 32 can be made of a waterproof material so that the expandable, immobilizing device 10 can be used in harsh environments such as in the rain, sleet, snow, mud, or chemical environments, without deterioration. A cotton stocking may be applied to the leg prior to placement of the expandable, immobilizing device 10 for comfort reasons. Alternatively, an absorbent material 77, such as, but not limited to a cotton material, cotton mesh, cotton gauze pads, absorbent polyester gauze pads may be adhesively fastened to the inner wall 20, see FIG. 5D, or to the compressible material pouch 32, see FIG. 6D.

The device may be made of radiolucent material to allow radiologic interpretation as to the status of the fracture of the inserted leg, thereby removing the need to remove the device prior radiologic detection steps. The device is designed to be MRI compliant. In surgical settings, the device can be designed as a pre and post-op dressing, reducing the time of surgery as the surgeon does not need to wrap a plaster splint or other surgical dressings.

The present invention further contemplates a kit. The kit composing one or more expandable, immobilizing device 10 in accordance with the present invention in a variety of sizes or all in the same size. A kit 78 may include one or more immobilizing devices 10 in the compressed state which are enclosed within an outer package structure 80. The outer package structure 80 preferably includes an outer package structure sealing member 82 such as an adhesive, to contain the immobilizing device 10 therein and prevent contamination. The kit may further include one or more straps, a cotton sleeve, and other materials that may be needed during rescue operations. Since the device can be positioned in a flattened state without causing damage, storage of multiple units is not a concern, even for places such as hospitals, emergency vehicles, or for teams traveling to remote or disaster locations, where space is at a premium. The kit may include pre-formed immobilizing device 10 in different sizes or shapes, or may be supplied as a generally flat sheet(s) to be configured as needed. FIG. 18 illustrates the immobilizing device 10 removed from outer package structure 80. The immobilizing device 10, which can have any of the features as described previously, has a first half 84 and a second half 86. Each half 84 and 86 contain portions which form the main body having a non-liner longitudinal center line, such as described for the shape of the first long arm and/or the second short arm, as described previously as 12 or 16. The halves 84 and 86 are constructed as mirror images when not folded, and can be positioned over a leg so the heal of the leg can be supported by or engaged with a recessed portion 82. The immobilizing device 10 may include a fold line 88 which forms a hinge. The fold line 88 preferably does not completely traverse the wall 30 (or double walls 18 and 20) so that the device lies in a generally flat configuration but can be easily bent over and positioned onto the leg prior to being secured by securing straps. As a result of the compression, excess material 88 (with or without a fold line) may cause a bubbling or extending of the expandable material 22 or compressible material pouch 32 prior in the compression state and prior to entering the non-compressed state.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A device for immobilizing fractures within an animal extremity comprising:
an expandable body formable into a device for immobilizing fractures within an animal extremity, said expandable body traversable from a first compressed position to a second non-compressed position.

2. The device for immobilizing fractures within a animal extremity according to claim 1 wherein said expandable body comprises a non-linear longitudinal center line that contours the shape of an extremity of an animal.

3. The device for immobilizing fractures within a animal extremity according to claim 2 wherein said non-linear longitudinal center line includes at least one curvature.

4. The device for immobilizing fractures within a animal extremity according to claim 2 wherein said non-linear longitudinal center line includes a plurality of curvatures.

5. The device for immobilizing fractures within a animal extremity according to claim 1 wherein said expandable body includes at least one reinforcement structure for providing support and stability to said expandable body.

6. The device for immobilizing fractures within a animal extremity according to claim 5 wherein said expandable body includes at least one reinforcement structure attached to the outer surface of said expandable body, said at least one reinforcement sized and shaped to contour at least a portion of said expandable body.

7. The device for immobilizing fractures within a animal extremity according to claim 1 further including at least one securing member.

8. The device for immobilizing fractures within a animal extremity according to claim 1 further includes a bottom support structure adapted to be secured to the bottom of said second portion.

9. The device for immobilizing fractures within a animal extremity according to claim 1 wherein said expandable body includes an opening sized to allow for insertion of an animal extremity therein and having a length that contours at least a portion of said expandable body.

10. The device for immobilizing fractures within a animal extremity according to claim 9 wherein said opening extends the length of said expandable body.

11. The device for immobilizing fractures within a animal extremity according to claim 1 wherein said expandable body has a first outer wall forming an exterior surface, a second inner wall for conforming to the shape of a mammalian extremity, and a compressible and expandable material in a compressed state there between.

12. The device for immobilizing fractures within a animal extremity according to claim 11 wherein said expandable body further includes an absorbent material.

13. The device for immobilizing fractures within a animal extremity according to claim 1 wherein said expandable body includes a single wall, and a compressible material pouch secured thereto having a compressible and expandable material in the compressed state.

14. The device for immobilizing fractures within a animal extremity according to claim 13 wherein said expandable body further includes an absorbent material.

15. The device for immobilizing fractures within a animal extremity according to claim 1 further containing contain one or more covered compression openings adapted to provide a mechanism for allowing the compressible and expandable material to expand from a compressed state to a non-compressed state.

16. The device for immobilizing fractures within a animal extremity according to claim 1 wherein said expandable body maintained in is stored as a substantially flat unit.

17. The device for immobilizing fractures within a animal extremity according to claim 16 wherein said expandable body consists of a first half and a second half which fold together to form a shaped expandable body configuration.

18. The device for immobilizing fractures within a animal extremity according to claim 16 wherein said expandable body includes a fold line.
